(19) United States
(12) Patent Application Publication
(10) Pub. No.: US 2019/0167572 A1
(43) Pub. Date: Jun. 6, 2019

(54) SUBCUTANEOUS IMPLANT-TYPE DEVICE FOR CELL TRANSPLANTATION THERAPY

(71) Applicant: TOHOKU UNIVERSITY, Miyagi (JP)

(72) Inventors: Toshiaki ABE, Miyagi (JP); Nobuhiro NAGAI, Miyagi (JP); Shinji YAMADA, Miyagi (JP); Masafumi GOTO, Miyagi (JP); Kozue IMURA, Miyagi (JP); Hirokazu KAJI, Miyagi (JP)

(73) Assignee: TOHOKU UNIVERSITY, Miyagi (JP)

(21) Appl. No.: 15/772,697

(22) PCT Filed: Nov. 7, 2016
(86) PCT No.: PCT/JP2016/082989

§ 371 Date: Jun. 27, 2018

(30) Foreign Application Priority Data
Nov. 6, 2015 (JP) 2015-218456

(51) Int. Cl. A61K 9/00; A61K 9/48; A61K 38/18

(52) U.S. Cl. CPC A61K 9/0024; A61K 9/4866; A61K 38/1825; A61K 9/4816

(57) ABSTRACT

A capsule device, which can induce vascular bed formation and formation of a space for cell transplantation in subcutaneous tissue in which the device is implanted, is provided. A sustained drug release capsule device, which is to be subcutaneously implanted for subcutaneous sustained release of an angiogenesis-inducing factor as well as formation of a vascular bed as a basis for cell transplantation and a space for cell transplantation in cell therapy, the capsule being made of a material which is not soluble in a biological body and does not adhere to a biological body, and having micropores for sustained release of the angiogenesis-inducing factor.

### TECHNICAL FIELD

The present invention relates to a capsule device which can be implanted into subcutaneous tissue. Specifically, the present invention relates to a capsule device, which is subcutaneously implanted for sustained release of an agent involved in angiogenesis at a certain sustained release rate for a long period of time so as to allow formation of a vascular bed and a space for cell transplantation.

### BACKGROUND ART

Organ transplantation has been a therapy for disorder of dysfunctional organs. Recently, studies have been conducted on cell transplantation therapy involving transplantation of cells of organs and the like for allowing the cells to exhibit the functions of the organs for treatment of various diseases.

Cell transplantation therapy is conducted by transplanting cells that constitute tissue to be treated or stem cells that can differentiate into such cells. As an example of cell transplantation therapy, a diabetes therapy involving islet cell transplantation or the like is performed. Unlike transplantation of organs such as pancreas, the cell transplantation therapy does not require a large-scale operation such as laparotomy. The therapy can be completed by transplanting cells within a short period of time.

As a technique for cell transplantation therapy, there is a report on a subcutaneous implant cell transplantation bag, which is characterized in that a permeable bag, which is formed with a polymer material, accommodates a sustained release carrier containing collagen and an angiogenesis-inducing factor (see Patent Literature 1). By filling the bag with cells to be transplanted and subcutaneously implanting the bag, it is possible to exhibit the effects of cell therapy. For example, such bag can be used as an artificial pancreas by filling it with islet cells or like.

In the case of cell therapy using this bag, it is required to allow a foreign matter to a biological body to be embedded in vivo for a long period of time.

### CITATION LIST

### Patent Literature

Patent Literature 1: JP Patent Publication (Kokai) No. 2001-299908 A

### SUMMARY OF INVENTION

### Technical Problem

Although cell transplantation therapy in subcutaneous tissue is safe because it is less invasive, as the degree of vascular distribution in subcutaneous tissue is low, supply of oxygen and nutrients to transplanted cell mass is insufficient, which is problematic.

It is an object of the present invention to provide a capsule device, which can induce vascular bed formation in subcutaneous tissue in which the device is implanted and formation of a space for cell transplantation. It becomes possible to directly transplant cells in subcutaneous tissue rich in blood vessels distributed as a result of implantation of the capsule device of the present invention so as to provide a vascular environment in which transplanted cells can survive. Accordingly, long-term survival of transplanted cells and the persistence of transplantation effects are expected.

### Solution to Problem

The present inventors made intensive studies with an aim to induce pretransplant vascular bed formation in the cell transplantation region as an auxiliary means for achieving minimally invasive subcutaneous islet cell transplantation into a patient, which will be an ultimate diabetes treatment therapy. In order to maintain the function of subcutaneously transplanted islet cells having hypovascular characteristics, it is important to form a vascular bed that serves as a basis for cell survival at the transplantation site from the early stage.

The present inventors created a capsule device, which is formed with triethylene glycol dimethacrylate (TEGDM) or polytetramethylene glycol diacrylate (PTMG) as a material and has void spaces for containing a drug and micropores for sustained release of the drug therein.

The present inventors introduced an angiogenesis-inducing factor into the device, subcutaneously implanted the device in animals, allowed the angiogenesis-inducing factor to be sustained-released for a given period of time, and removed the implanted device. The present inventors found that a vascular bed is formed in the subcutaneous tissue where the device has been implanted, and a space into which cells can be transplanted is further formed on the vascular bed. This has led to the completion of the present invention.

In other words, the present invention is described as follows.

[1] A sustained drug release capsule device, which is to be subcutaneously implanted for subcutaneous sustained release of an angiogenesis-inducing factor as well as formation of a vascular bed as a basis for cell transplantation and a space for cell transplantation in cell therapy, wherein the capsule is made of a material which is not soluble in a biological body and does not adhere to a biological body, and has micropores for sustained release of the angiogenesis-inducing factor.
[2] The sustained drug release capsule device of [1], wherein the material which is not soluble in a biological body and does not adhere to a biological body is selected from the group consisting of triethylene glycol dimethacrylate (TEGDM), tetraethylene glycol dimethacrylate, polytetramethylene glycol diacrylate (PTMG), polypropylene glycol diacrylate, isostearyl acrylate, urethane acrylate, methoxy polyethylene glycol acrylate, 2-hydroxy-3-acryloyloxy propyl methacrylate, dipropylene glycol diacrylate, tripropylene glycol diacrylate, 1,9-nonanediol diacrylate, 1,6-hexanediol diacrylate, 1,10-decanediol diacrylate, tricyclodecane dimethanol diacrylate, propoxylated bisphenol A diacrylate, 9,9-bis[4-(2-acryloyloxyethoxy)phenyl]fluorene, ethoxylated bisphenol A diacrylate, propoxylated ethoxylated bisphenol A diacrylate, and 2-hydroxy-3-acryloyloxy propyl methacrylate.
[3] The sustained drug release capsule device of [1] or [2], wherein the angiogenesis-inducing factor is selected from the group consisting of a basic fibroblast growth factor (bFGF), an acidic fibroblast growth factor (aFGF), a vascular endothelial growth factor (VEGF), a platelet-derived growth factor, a transforming growth factor-β (TGF-β), a hepatocyte growth factor (HGF), osteonectin, and angiopoietin.
[4] The sustained drug release capsule device of any one of [1] to [3], which encapsulates the angiogenesis-inducing factor.
[5] The sustained drug release capsule device of [4], wherein the angiogenesis-inducing factor is mixed and pelletized with a water-soluble polymer.

The present description includes part or all of the contents as disclosed in the description and/or drawings of Japanese Patent Application No. 2015-218456, which is a priority document of the present application.

### Advantageous Effects of Invention

The bFGF-containing capsule device of the present invention has been confirmed to be effective for sustained release of bFGF in in vitro tests. Further, it has been confirmed that bFGF exhibits its effects (cell growth and angiogenesis) in the tissue surrounding the device in a case in which the device was subcutaneously transplanted in rats. In addition, the transplanted device does not adhere to subcutaneous tissue, and thus, it can be easily removed. Therefore, the space occupied by the device can be used as a space for cell transplantation in the subsequent subcutaneous transplantation of cells. The issue surrounding the site of cell transplantation is rich in blood vessels, which facilitates survival of transplanted cells and enables the maintenance of cell functions.

### BRIEF DESCRIPTION OF DRAWINGS

FIG. 1 includes photographs showing the appearance of the capsule device made of PTMG according to the present invention. FIG. 1A shows the shape of the capsule device having micropores. FIG. 1B shows the capsule device in a state of being bent with fingers. FIG. 1C shows the capsule device filled with agarose gel in a void space therein.

FIG. 2 shows a cross-sectional view (A) and a top view (B) of the capsule device of the present invention.

FIG. 3 shows graphs indicating characteristics of sustained release of bFGF from the capsule device of the present invention.

FIG. 4 includes photographs showing the state of rat subcutaneous tissue in which a capsule device formed with TEGDM (containing a placebo without bFGF) according to the present invention was transplanted and the state of rat subcutaneous tissue from which the device was removed.

FIG. 5 includes photographs showing the state of rat subcutaneous tissue in which a capsule device formed with TEGDM (containing bFGF) according to the present invention was transplanted and the state of rat subcutaneous tissue from which the device was removed.

FIG. 6 includes photographs showing the state of rat subcutaneous tissue in which a capsule device formed with PTMG (containing a placebo without bFGF) was transplanted and the state of rat subcutaneous tissue from which the device was removed.

FIG. 7 includes photographs showing the state of rat subcutaneous tissue in which a capsule device formed with PTMG (containing bFGF) was transplanted and the state of rat subcutaneous tissue from which the device was removed.

FIG. 8 includes photographs of sections of untreated rat subcutaneous tissue.

FIG. 9 includes photographs of sections of rat subcutaneous tissue in which a TEGDM placebo device was implanted.

FIG. 10 includes photographs of sections of rat subcutaneous tissue surrounding a device (on one side of the device in contact with the skin) which is a subcutaneously implanted bFGF/TEGDM device.

FIG. 11 includes photographs of sections of rat subcutaneous tissue surrounding a device (on one side of the device in contact with the muscle) which is a subcutaneously implanted bFGF/TEGDM device.

### DESCRIPTION OF EMBODIMENTS

Hereinafter, the present invention is described in detail.

The present invention concerns a capsule device, which is an implant drug device that can be implanted in subcutaneous tissue. The capsule device of the present invention has a void space that functions as a reservoir for encapsulating a substance. Therefore, it is also referred to as a “device reservoir” in some cases. In the present invention, subcutaneous embedment of a capsule device is also expressed as “subcutaneous implantation.”

The capsule device encapsulates an angiogenesis-inducing factor and has micropores (micro-size pores) on its surface for sustained-release of the angiogenesis-inducing factor encapsulated therein. In a case in which the capsule device is subcutaneously implanted, it serves as a DDS (drug delivery system) device for subcutaneous sustained release of the angiogenesis-inducing factor. The device of the present invention can also be referred to as a “sustained drug release capsule device.”

In vivo sustained release of the angiogenesis-inducing factor encapsulated in the device from micropores formed on the device surface enables induction of vascular bed formation in subcutaneous tissue in which the device is implanted. A material for the device is elastic and highly biocompatible, and thus, the device does not adhere to surrounding subcutaneous tissue, making it possible to readily remove the device. A space in which cells can be transplanted is formed after the removal of the device. This makes it possible to directly transplant cells in subcutaneous tissue rich in blood vessels distributed as a result of device implantation so as to provide a vascular environment in which transplanted cells can survive.

In other words, the capsule device of the present invention is subcutaneously implanted to function to allow sustained release of an angiogenesis-inducing factor, thereby induce vascular bed formation in subcutaneous tissue and further inducing formation a space for cell transplantation.

The term “vascular bed” used herein refers to tissue in which a vascular network has been formed, which is used as a basis for cell transplantation.

The capsule device of the present invention is a box-type device having a void space which serves as a reservoir therein. The term “box-type” used herein means a shape that allows encapsulation of a substance. Preferably, the capsule device has a top face, a bottom face, and a lateral face, and it is substantially disk-shaped such that the top face and the bottom face each have a substantially circular shape or it is substantially cube-shaped such that the top face and the bottom face each have a substantially rectangular shape. In addition, as long as it has a surface for sustained release of a substance encapsulated therein, the shape thereof is not limited and thus it may be flat, substantially spherical, or substantially cylindrical.

In a case in which the capsule device is substantially disk-shaped, the diameter of the substantially circular top or bottom face is several tens of millimeters, preferably 5 to 50 mm, more preferably 10 to 30 mm, and particularly preferably 20 to 30 mm. In a case in which the capsule device is substantially cube-shaped, the length of a side of the substantially rectangular top or bottom face is several tens of millimeters, preferably 5 to 50 mm, more preferably 10 to 30 mm, and particularly preferably 20 to 30 mm. The surface area of the top or bottom face is approximately 20 mm² to 2000 mm² and the thickness thereof is several millimeters, preferably 1 to 7 mm, and more preferably 2 to 5 mm.

The above sizes are merely examples. Therefore, it is possible to appropriately design the size of the capsule device depending on the size of a space for cell transplantation that is subcutaneously formed.

The encapsulated angiogenesis-inducing factor is sustained-released from micropores formed on either one of or both of the top face and the bottom face. The top face and the bottom face each have a thin-film shape for the purpose of sustained release of a substance. The thickness thereof is 100 to 1000 μm. The lateral face needs to have a certain thickness in order to maintain the box-type shape. The thickness thereof is about 500 to 2000 μm.

Either one of or both of the top face and the bottom face through which the encapsulated substance is sustained-released have micropores for sustained release of the substance. The number of micropores is 1 to 200 and preferably 5 to 100. The diameter of micropores is several micrometers.

The diameter or number (density) of micropores can be appropriately designed depending on the molecular weight of an encapsulated angiogenesis-inducing factor to be sustained-released and a preferable sustained release rate.

The capsule device of the present invention has micropores, thereby achieving sustained release of the encapsulated angiogenesis-inducing factor with certainly. Thus, it is possible to achieve a desired sustained release rate by adjusting the number of micropores.

The capsule device of the present invention can be produced by preparing two box-type parts each having a recess for accommodating a substance and joining the two parts such that the recesses form a closed space. Each part is sometimes referred to as a “reservoir.”

As a material for the capsule device of the present invention, a material which does not adhere to a biological body needs to be used. The material which does not adhere to a biological body is a substance that is not dissolved in vivo and does not adhere or bind to biological tissue. For example, since gelatin-based hydrogel, agarose gel, or the like may be dissolved in vivo, it can be used as a material with which a substance to be encapsulated is impregnated whereas it cannot be used as a material for the capsule device. In addition, it is difficult to allow a material that may be dissolved in vivo to form a space for cell transplantation.

For example, as a material for the capsule device, a photocurable resin that is cured when irradiated with UV light can be used. An example of a photocurable resin is a polymer of an ethylene glycol monomer, such as, triethylene glycol dimethacrylate (TEGDM) or tetraethylene glycol dimethacrylate. Further, examples of such resin that can be used include polytetramethylene glycol diacrylate (PTMG), polypropylene glycol diacrylate, isostearyl acrylate, urethane acrylate, methoxy polyethylene glycol acrylate, 2-hydroxy-3-acryloyloxy propyl methacrylate, dipropylene glycol diacrylate, tripropylene glycol diacrylate, 1,9-nonanediol diacrylate, 1,6-hexanediol diacrylate, 1,10-decanediol diacrylate, tricyclodecane dimethanol diacrylate, propoxylated bisphenol A diacrylate, 9,9-bis[4-(2-acryloyloxyethoxy)phenyl]fluorene, ethoxylated bisphenol A diacrylate, propoxylated ethoxylated bisphenol A diacrylate, and 2-hydroxy-3-acryloyloxy propyl methacrylate. Preferable resins are triethylene glycol dimethacrylate (TEGDM) and polytetramethylene glycol diacrylate (PTMG). TEGDM is less elastic than PTMG. However, TEGDM is highly biocompatible and excellent as a material for the capsule device. In addition, PTMG is highly elastic and excellent as a material for the capsule device. These materials are elastic enough to be subcutaneously implanted in a convenient manner. In addition, the materials do not adhere to biological tissue and are not dissolved or destroyed in vivo. This makes it possible to readily remove the capsule device even after the capsule device has been subcutaneously implanted for a certain period of time, thereby allowing formation of a subcutaneous space for cell transplantation. Note that since PEG such as polyethylene glycol dimethacrylate (PEGDM), polyethylene glycol methacrylate (PEGMA), or polyethylene glycol diacrylate (PEGDA) tends to be easily destroyed, it is not a suitable material for the capsule device.

In order to cure a photocurable resin, a photopolymerization initiator may be used. As the photopolymerization initiator used herein, a photopolymerization initiator known in the art can be appropriately selected depending on the wavelength of a light source to be used. Examples of such photopolymerization initiator include 2-hydroxy-2-methyl-propiophenone, 4′-isopropyl-2-hydroxy-2-methyl-propiophenone, 1-hydroxycyclohexyl phenyl ketone, 2,2-diethoxyacetophenone, benzyl methyl ketal, benzyl-β-methoxyethyl acetal, benzoin(2-phenyl-2-hydroxyacetophenone), and benzoin alkyl ether. The intensity of UV light used for photocuring is 1 to 20 mW/cm², and irradiation may be performed for 1 to 5 minutes.

The box-type part can be prepared by placing a mixture of the above material and a photopolymerization initiator in a mold, irradiating the mixture with UV light for curing, and removing the cured product from the mold. An angiogenesis-inducing factor to be sustained-released is placed in the recesses of two such box-type parts. Then, the two parts can be joined by photocuring. As the mold, for example, a mold formed with polydimethylsiloxane in a three-dimensional shape can be used.

Micropores can be formed by providing needle-like protrusions on the upper surface and/or the lower surface of a mold for producing parts of a capsule device. Alternatively, it is possible to prepare a capsule device or parts thereof and then perforate the capsule device or the parts thereof using a needle, a laser beam, or the like.

It can also be said that the capsule device of the present invention is a device composed of a reservoir containing an angiogenesis-inducing factor and a sustained release film from which the angiogenesis-inducing factor is sustained-released.

The capsule device of the present invention is elastic enough to be easily bent with fingers. Therefore, even when it is subcutaneously implanted, the risk of being destroyed is small. Further, the capsule device of the present invention is a drug reservoir-type DDS device that enables a long-term sustained release with suppressed initial burst.

The angiogenesis-inducing factor is not particularly limited as long as it can induce angiogenesis. However, examples thereof include a basic fibroblast growth factor (bFGF), an acidic fibroblast growth factor (aFGF), a vascular endothelial growth factor (VEGF), a platelet-derived growth factor, a transforming growth factor-β (TGF-β), a hepatocyte growth factor (HGF), osteonectin, and angiopoietin. It is also possible to allow a substance that can produce or express an angiogenesis-inducing factor to be contained. Examples of such substance include an expression vector containing DNA that encodes the angiogenesis-inducing factor and cells that can produce and secrete the angiogenesis-inducing factor. When an angiogenesis-inducing factor is produced or secreted in such case, it allows the capsule device to encapsulate the angiogenesis-inducing factor. Therefore, it can be referred to as “encapsulation of angiogenesis-inducing factor” according to the present invention.

Further, the capsule device may contain an immunosuppressant together with the angiogenesis-inducing factor in cell transplantation after the formation of a vascular bed and a space for cell transplantation in order to prevent rejection in the case of allogeneic transplantation. Examples of an immunosuppressant include cyclosporine, tacrolimus, sirolimus and derivatives thereof.

It is preferable to use an angiogenesis-inducing factor to be contained in the capsule device with a water-soluble polymer gel for sustained release. The angiogenesis-inducing factor is pelletized by being mixed with a polymer gel. It is said that the angiogenesis-inducing factor is pelletized with a polymer gel according to the present invention. Examples of a gel for sustained release include agarose gel, gelatin hydrogel, and polyethylene glycol dimethacrylate, which are not biologically toxic. It is possible to pelletize an angiogenesis-inducing factor with a polymer gel by a method known in the art. For example, in a case in which agarose gel is used, an agarose powder is dissolved by heating in a buffer and mixed with an angiogenesis-inducing factor, the mixture is placed in a capsule device and left for cooling, followed by gelation. In such case, when an angiogenesis-inducing factor is an unstable substance, a substance that can stabilize an angiogenesis-inducing factor and aid activity of the factor may be added. For example, in a case in which heparin is added, it binds to an angiogenesis-inducing factor such as bFGF so that the angiogenesis-inducing factor is protected and stabilized.

The period of sustained release of an angiogenesis-inducing factor for vascular bed formation is 5 days to several weeks, preferably 7 to 15 days, and more preferably 10 to 12 days. The amount of an angiogenesis-inducing factor to be sustained-released is preferably approximately 1 to 10 μg per day. However, the necessary amount for sustained release and the sustained release rate are determined based on the required size of a vascular bed and the like. It is possible to adjust the amount for sustained release and the sustained release rate based on the capsule device size and the number and size of micropores on the capsule device surface. The capsule device of the present invention is subcutaneously implanted during the above period such that a space for the subsequent cell transplantation is formed.

A vascular bed having a vascular network is formed as a result of sustained release of an angiogenesis-inducing factor in subcutaneous tissue in which a capsule device has been implanted, and a space is formed at a site where the capsule device has been implanted. Thereafter, cells or tissue for transplantation can be transplanted into the space.

Examples of such cells include islet cells, which can be used in the treatment of diabetes.

Transplanted cells are supplied with nutrients from the vascular network of a vascular bed so as to grow, and hormones and physiologically active substances produced and secreted by the transplanted cells are incorporated into the vascular network of a vascular bed so as to be systemically delivered.

In addition, a space including a formed vascular bed can also be used as a pathway for administering a protein agent such as an antibody. For example, a protein such as an antibody may be administered into a space including a formed vascular bed or a DDS device containing a protein such as an antibody may be transplanted.

### EXAMPLES

Hereinafter, the present invention is specifically described with reference to the following Examples. However, the present invention is not limited to these

### Example 1: Production of a Sustained Drug Release Capsule Device (a Device for Sustained Release of bFGF)

### 1.1 Preparation of a Reservoir

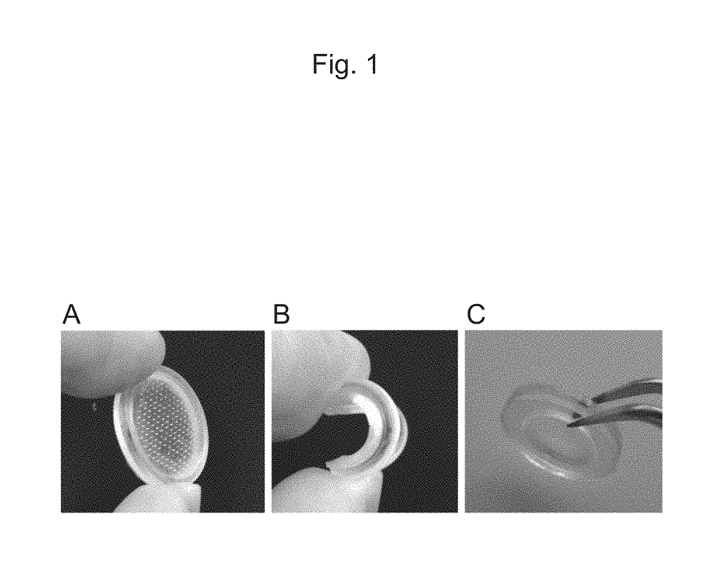

2-HMP (2-hydroxy-2-methyl-propiophenone) was added as a curing initiator in an amount of 0.2 mL to 10 mL of a prepolymer of PTMG (polytetramethylene glycol diacrylate) or TEGDM (triethylene glycol dimethacrylate), followed by mixing and deaeration (0.08 MPa, 10 minutes). Subsequently, 800 μL of the liquid mixture was poured into a concave PDMS (polydimethylsiloxane) mold, a convex PDMS mold was placed thereon, and PTMG (or TEGDM) was cured by irradiation with UV light (11.6 mW/cm²) for 40 seconds. Then, a PTMG (or TEGDM) reservoir was obtained as one part of a device by removing the cured product from the PDMS mold. FIG. 1A shows the appearance of the reservoir. The reservoir prepared with PTGM is elastic enough to be easily bent with fingers as shown in FIG. 1B.

### 1.2 Preparation of bFGF/Heparin-Containing Agarose Gel

An agarose powder having a low melting point in an amount of 3.2 g was placed in 100 mL of phosphate buffered saline (PBS) and dissolved by heating at 121° C. for 15 minutes (3.2% agarose solution). Thereafter, the solution was incubated in a warm water bath at 42° C. to prevent gelation. A 100 μg/mL bFGF solution (50 μL) and a 1000 U/mL heparin solution (25 μL) were mixed and allowed to stand still in a warm bath at 42° C. for 5 minutes. The liquid mixture was mixed with 125 μL of the 3.2% agarose solution. The mixture was poured in an amount of 200 μL into the concave PDMS mold and allowed to stand still at 4° C. for 10 minutes for gelation.

### 1.3 Assembly of a Device

The bFGF/heparin-containing 2% agarose gel prepared in 1.2 was inserted into the PTMG (or TEGDM) reservoir prepared in 1.1. Thereafter, the reservoir was covered with a PTMG (or TEGDM) reservoir of the same shape and irradiated with UV light (11.6 mW/cm²) for 240 seconds such that the PTMG (or TEGDM) reservoirs were bonded to each other. Thus, a PTMG (or TEGDM) capsule device encapsulating bFGF/heparin-containing agarose gel was assembled. FIG. 1C shows the appearance of the capsule device filled with bFGF/heparin-containing agarose gel.

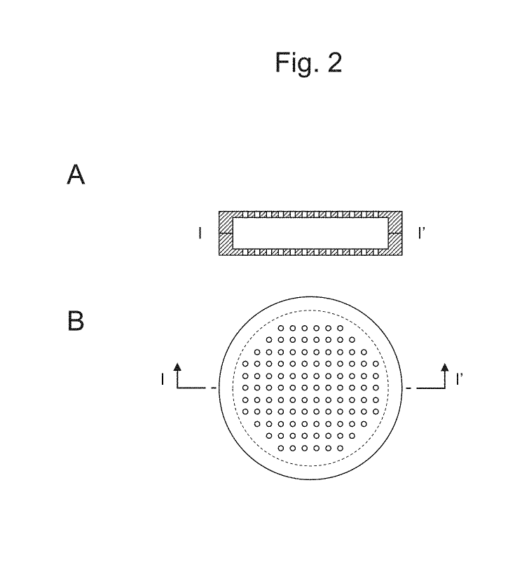

FIG. 2 schematically illustrates the capsule device of the present invention.

### Example 2: Test of Sustained Release of bFGF from the Capsule Device

bFGF sustained release TEGDM devices (one with 57 micropores and one with 5 micropores formed by the PDMS mold on the reservoir bottom) prepared in 1.3 and bFGF/heparin-containing agarose gel prepared in 1.2 were separately placed in 50 mL-volume polypropylene tubes, and 10 mL of PBS was poured thereinto. Thereafter, the tubes were allowed to stand still at 37° C. during which PBS was recovered and replaced over time. The bFGF concentration was determined by enzyme-linked immunosorbent assay (ELIS A).

Sampling was conducted 1, 3, 12, 24, 48, 72 and 96 hours later for agarose gel alone. For each TEGDM device, sampling was conducted 1, 3, 12, 24, 48, 72, 96, 120 and 144 hours later.

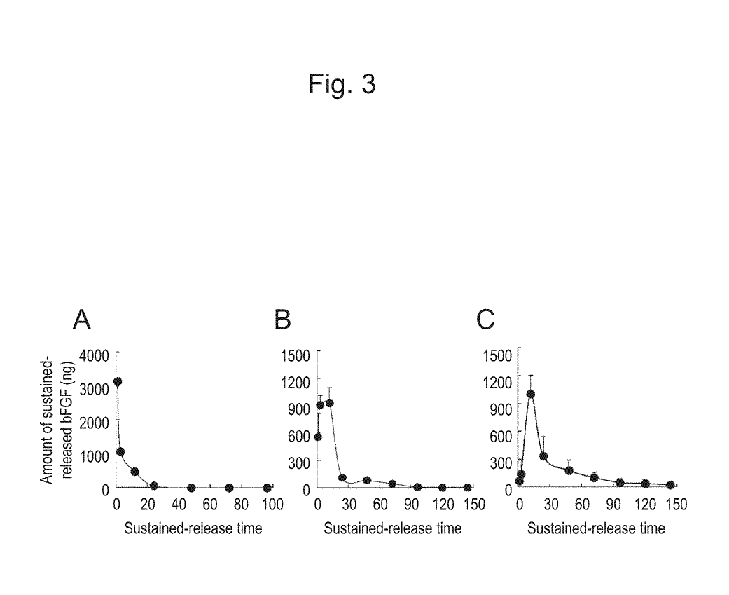

FIG. 3 indicates the results. FIG. 3A indicates the amount of bFGF released from agarose gel alone. FIG. 3B indicates the amount of bFGF released from the device having 57 pores on each side (114 pores on both sides in total). FIG. 3C indicates the amount of bFGF released from the device having 5 pores on each side (10 pores on both sides in total). FIG. 3 suggests that bFGF is released transiently in the case of agarose gel alone, while on the other hand, encapsulation of agarose containing bFGF with a capsule device having micropores formed with a photocurable resin such as TEGDM results in sustained release of bFGF.

### Example 3: Device Implantation Test

### 3-1. TEGDM Placebo Device Implantation Test

Healthy SD rats (male) with a weight of approximately 250 g were anesthetized with a ketamine-xylazine liquid mixture and the lateral region of each rat was shaved. Thereafter, the skin of the region was disinfected, and an incision was made thereon, and the skin was sutured after subcutaneously inserting a TEGDM placebo device (prepared by replacing 50 μL of a bFGF solution and 25 μL of a heparin solution by 75 μL of PBS during preparation of gel in 1.2). The suture site was incised 8 days after implantation, and subcutaneous tissue around the device was observed. In addition, a portion of the subcutaneous tissue around the device was cut out, the portion was fixed with 4% paraformaldehyde and paraffin-embedded. Histological findings were observed by HE staining.

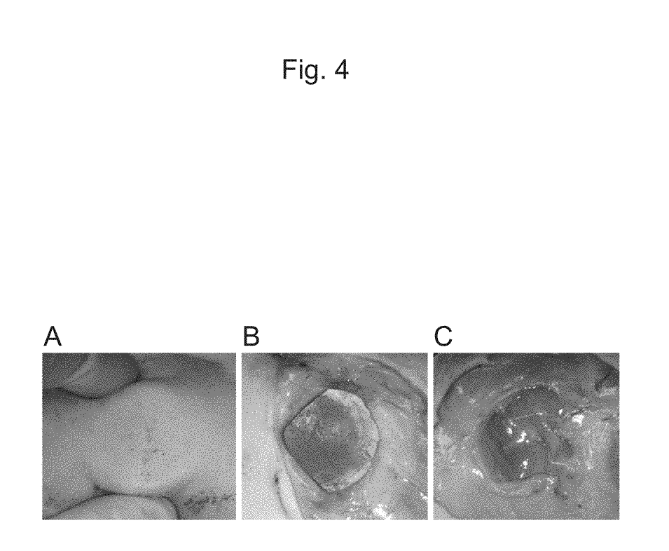

FIG. 4 indicates the results. FIG. 4A shows the appearance of the skin 8 days after subcutaneous transplantation of the device. FIG. 4B shows the state of the tissue surrounding the device 8 days after transplantation. FIG. 4C shows the state of the subcutaneous tissue after the removal of the device. Since there is no adhesion of the tissue surrounding the device, the device can be easily removed. In addition, since a space is formed after the removal of the device, it is possible to transplant cells into the space portion.

### 3-2. bFGF/TEGDM Device Implantation Test

A TEGDM device for sustained release of bFGF produced in the manner described in Example 1 was subcutaneously implanted into SD rats in the manner described in 3-1. Observation was conducted in the same manner.

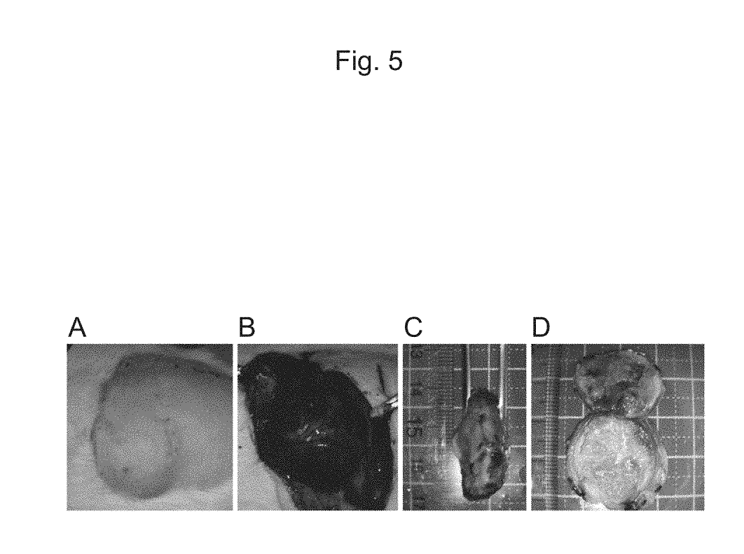

FIG. 5 indicates the results. FIG. 5A shows the appearance of the skin 8 days after subcutaneous transplantation of a capsule device containing bFGF. FIG. 5B shows the state of the tissue surrounding the device 8 days after transplantation. The region surrounding the device became hyperemic and was covered with grown cells. This result indicates that a vascular bed was probably formed as a result of sustained release of bFGF. FIG. 5C shows the state of the tissue section (formalin-fixed) surrounding the device. FIG. 5D shows the inside of the tissue section (formalin-fixed) after removal of the device. A space is formed inside the tissue section.

### 3-3. PTMG Placebo Device Implantation Test

A PTMG placebo device was subcutaneously implanted into SD rats in the manner described in 3-1. Observation was conducted in the same manner.

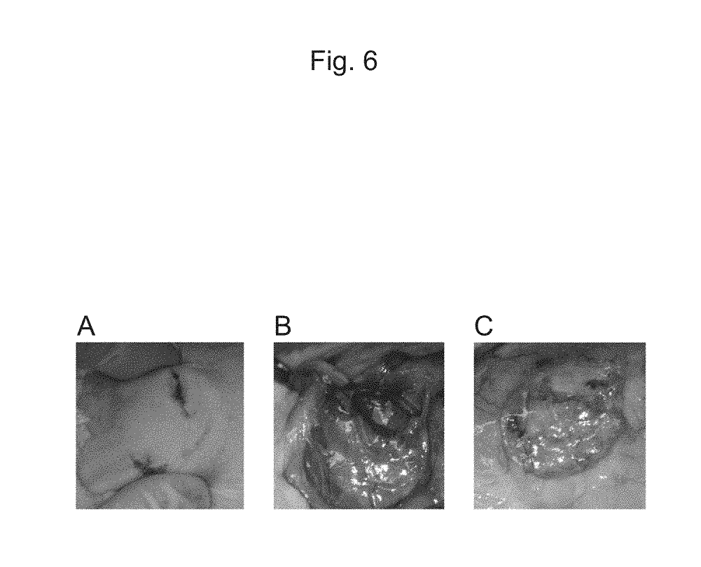

FIG. 6 indicates the results. FIG. 6A shows the appearance of the skin 8 days after subcutaneous transplantation of the device. FIG. 6B shows the state of the tissue surrounding the device 8 days after transplantation. Capsular formation and some hydrophobic degeneration are observed around the device. FIG. 6C shows the state of the subcutaneous tissue after the removal of the device. Since there is no adhesion of the tissue surrounding the device, the device can be easily removed. In addition, since a space is formed after the removal of the device, it is possible to transplant cells into the space portion.

### 3-4. bFGF/PTMG Device Implantation Test

A PTMG device for sustained release of bFGF produced in the manner described in Example 1 was subcutaneously implanted into SD rats in the manner described in 3-1. Observation was conducted in the same manner.

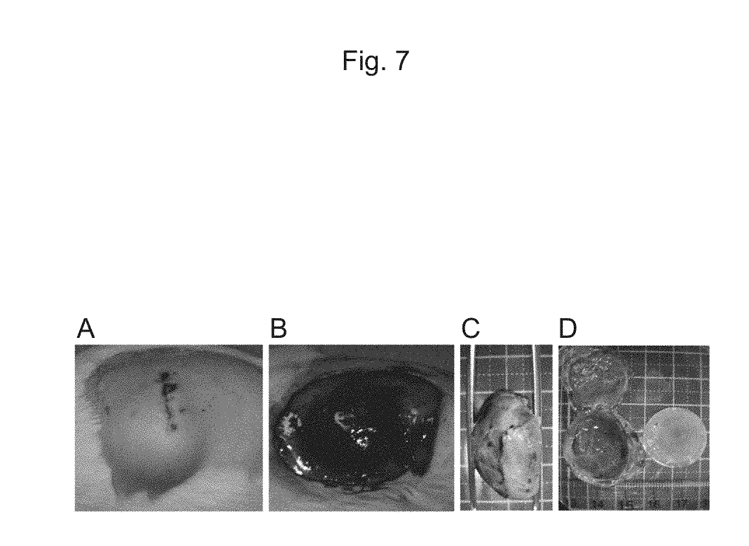

FIG. 7 indicates the results. FIG. 7A shows the appearance of the skin 8 days after subcutaneous transplantation of the capsule device. FIG. 7B shows the state of the tissue surrounding the device 8 days after transplantation. The region surrounding the device became hyperemic and was covered with grown cells. This result indicates that a vascular bed was probably formed as a result of sustained release of bFGF. FIG. 7C shows the tissue section (formalin fixed) surrounding the device. FIG. 7D shows the inside of the tissue section (formalin fixed) after removal of the device. A space is formed inside the tissue section.

### 3-5. Observation of Vascular Distribution in Subcutaneous Tissue

Rats in which the bFGF/TEGDM device was implanted, a rat in which the TEGDM placebo device was implanted, and an untreated control mouse were subjected to HE (hematoxylin-eosin) staining for observation of subcutaneous vascular distribution.

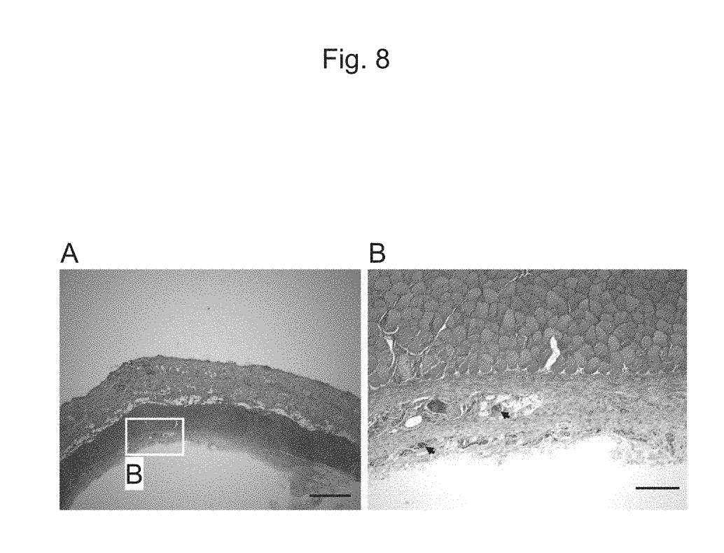

FIG. 8 includes photographs of subcutaneous tissue sections of an untreated rat. FIG. 8A shows a cross-section of the abdominal skin of the rat (1-mm scale). FIG. 8B is an enlarged view of subcutaneous tissue shown in the frame in FIG. 8A (200-μm scale). Each arrow indicates the vascular position in FIG. 8B. As shown in the figures, there is substantially no subcutaneous vascular distribution in the untreated rat.

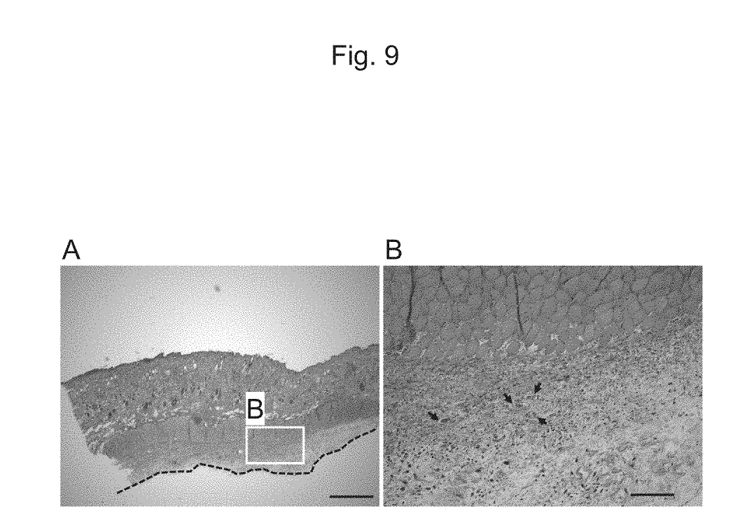

FIG. 9 includes photographs of sections of subcutaneous tissue of the rat in which a TEGDM placebo device produced in 3-2 was implanted. FIG. 9A shows a cross-section of the skin surrounding the device (1-mm scale). A broken line indicates a side in contact with the device in FIG. 9A. FIG. 9B is an enlarged view of subcutaneous tissue shown in the frame in FIG. 9A (200-μm scale). Each arrow indicates the vascular position in FIG. 9B. As shown in the figures, there is substantially no subcutaneous vascular distribution in the rat in which the TEGDM placebo device was implanted.

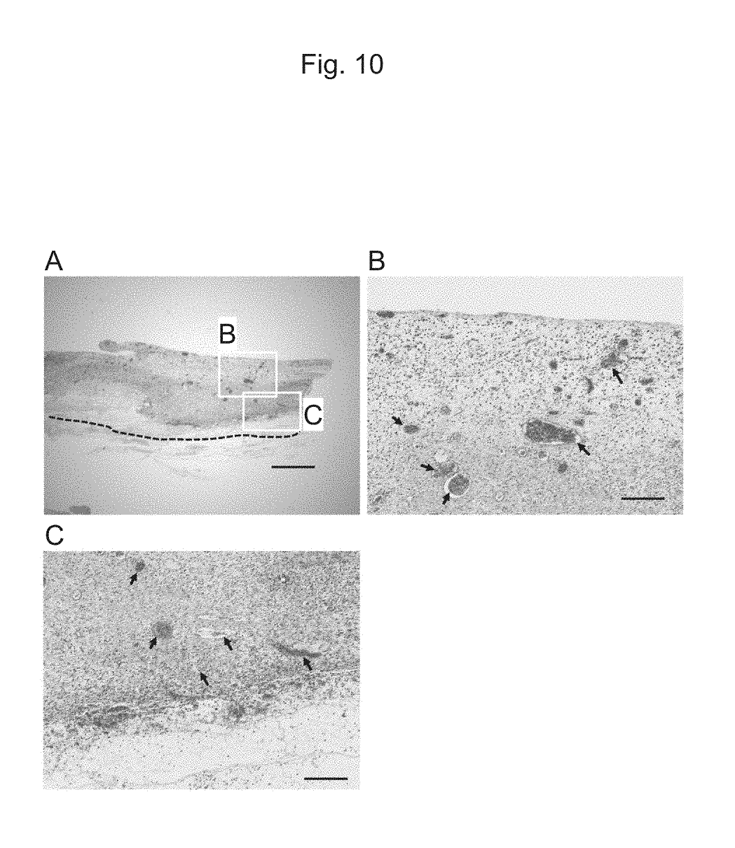

FIG. 10 includes photographs of sections of device-surrounding subcutaneous tissue (on a skin side in contact with the device) of the rat in which a bFGF/TEGDM device produced in 3-2 was implanted. FIG. 10A shows a cross-section of the skin surrounding the device (1-mm scale). A broken line indicates a side in contact with the device in FIG. 10A. FIG. 10B is an enlarged view of subcutaneous tissue (shown in the frame in FIG. 10A) on the skin side (200-μm scale). Each arrow indicates the vascular position in FIG. 10B. FIG. 10C is an enlarged view of subcutaneous tissue (shown in the frame in FIG. 10A) on the device side (200-μm scale). Each arrow indicates the vascular position in FIG. 10C. As shown in the figures, many capillary vessels are formed.

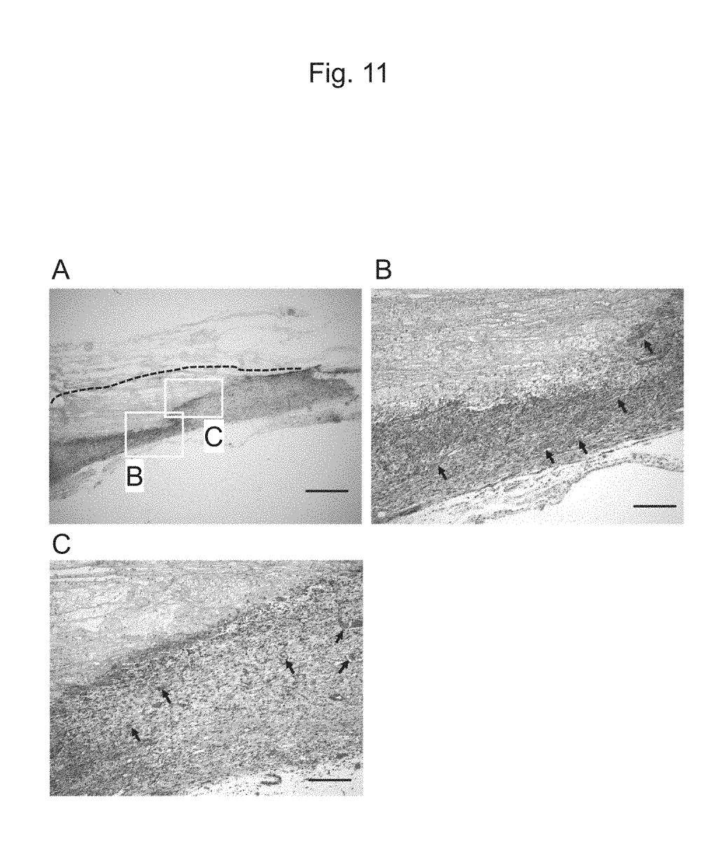

FIG. 11 includes photographs of sections of device-surrounding subcutaneous tissue (on a muscle side in contact with the device) of the rat in which a bFGF/TEGDM device produced in 3-2 was implanted. FIG. 11A shows a cross-section of the skin surrounding the device (1-mm scale). A broken line indicates a side in contact with the device in FIG. 11A. FIG. 11B is an enlarged view of subcutaneous tissue (shown in the frame in FIG. 11A) on the skin side (200-μm scale). Each arrow indicates the vascular position in FIG. 11B. FIG. 11C is an enlarged view of subcutaneous tissue (shown in the frame in FIG. 11A) on the device side (200-μm scale). Each arrow indicates the vascular position in FIG. 11C. As shown in the figures, many capillary vessels are formed.

### INDUSTRIAL APPLICABILITY

The capsule device of the present invention can be used in the fields of cell transplantation therapy and regenerative medicine.

All publications, patents and patent applications cited in the present description are incorporated herein by reference in their entirety.

1. A sustained drug release capsule device, which is to be subcutaneously implanted for subcutaneous sustained release of an angiogenesis-inducing factor as well as formation of a vascular bed as a basis for cell transplantation and a space for cell transplantation in cell therapy, wherein the capsule is made of a material which is not soluble in a biological body and does not adhere to a biological body, and has micropores for sustained release of the angiogenesis-inducing factor.

2. The sustained drug release capsule device of claim 1, wherein the material which is not soluble in a biological body and does not adhere to a biological body is selected from the group consisting of triethylene glycol dimethacrylate (TEGDM), tetraethylene glycol dimethacrylate, polytetramethylene glycol diacrylate (PTMG), polypropylene glycol diacrylate, isostearyl acrylate, urethane acrylate, methoxy polyethylene glycol acrylate, 2-hydroxy-3-acryloyloxy propyl methacrylate, dipropylene glycol diacrylate, tripropylene glycol diacrylate, 1,9-nonanediol diacrylate, 1,6-hexanediol diacrylate, 1,10-decanediol diacrylate, tricyclodecane dimethanol diacrylate, propoxylated bisphenol A diacrylate, 9,9-bis[4-(2-acryloyloxyethoxy)phenyl]fluorene, ethoxylated bisphenol A diacrylate, propoxylated ethoxylated bisphenol A diacrylate, and 2-hydroxy-3-acryloyloxy propyl methacrylate.

3. The sustained drug release capsule device of claim 1, wherein the angiogenesis-inducing factor is selected from the group consisting of a basic fibroblast growth factor (bFGF), an acidic fibroblast growth factor (aFGF), a vascular endothelial growth factor (VEGF), a platelet-derived growth factor, a transforming growth factor-β (TGF-β), a hepatocyte growth factor (HGF), osteonectin, and angiopoietin.

4. The sustained drug release capsule device of claim 1, which encapsulates the angiogenesis-inducing factor.

5. The sustained drug release capsule device of claim 4, wherein the angiogenesis-inducing factor is mixed and pelletized with a water-soluble polymer.
